(19) United States
(12) Patent Application Publication
(10) Pub. No.: US 2017/0157281 A1
(43) Pub. Date: Jun. 8, 2017

(54) ATOMIZING AROMA DIFFUSER

(71) Applicant: Xiaoyuan Ma, Shenzhen (CN)

(72) Inventor: Xiaoyuan Ma, Shenzhen (CN)

(21) Appl. No.: 15/350,111

(22) Filed: Nov. 14, 2016

(30) Foreign Application Priority Data
Dec. 4, 2015 (CN) 201520989271.4

(51) Int. Cl. A61L 9/14

(52) U.S. Cl. CPC A61L 9/14; A61L 2209/12; A61L 2209/134

(57) ABSTRACT

The present invention discloses an atomizing aroma diffuser which comprises an outer cover, a hollow diffuser body and a base, wherein the outer cover covers the outside of the hollow diffuser body; the upper surface of the outer cover is provided with a vent hole; the base is connected with the lower end of the hollow diffuser body; the base is provided with a fan which is electrically connected with a circuit board; a light-emitting diode (LED) light sheet is further integrated on the circuit board; a plastic partition plate with the hollow middle is arranged in the hollow diffuser body and divides the hollow diffuser body into an upper part and a lower part; a ceramic atomizing sheet is installed on the middle hollow part of the plastic partition plate and connected with the circuit board; one side wall of the hollow diffuser body is provided with an exhaust channel, and the exhaust channel penetrates the plastic partition plate; and a cover with a vent hole is further arranged above the hollow diffuser body. The electric aroma diffuser which is simple in structure and has an atomization function and colored light is provided. The electric aroma diffuser is attractive and practical and can create a cozy atmosphere.

### CROSS-REFERENCE TO RELATED APPLICATIONS

This application claims priority benefit of Chinese Patent Application No. 201520989271.4 with a filing date of Dec. 4, 2015. The content of the aforementioned application, including any intervening amendments thereto, are incorporated herein by reference.

### TECHNICAL FIELD

The present invention relates to an electric aroma diffuser, and in particular to an atomizing aroma diffuser.

### BACKGROUND OF THE PRESENT INVENTION

What are used in conventional aroma diffusers are mostly flames, which lead to hidden danger easily; meanwhile, smoke is generated to pollute air at home. Some existing electric aroma diffusers are complex in structure and not attractive.

### SUMMARY OF PRESENT INVENTION

In order to solve the problems in the prior art, the present invention provides an electric aroma diffuser which is simple in structure and has an atomization function and colored light. The electric aroma diffuser is attractive and practical and can create a comfortable atmosphere.

The present invention can be implemented through the following technical solution:

an atomizing aroma diffuser comprises an outer cover, a hollow diffuser body and a base, wherein the outer cover covers the outside of the hollow diffuser body; the upper surface of the outer cover is provided with a vent hole; the base is connected with the lower end of the hollow diffuser body; the base is provided with a fan which is electrically connected with a circuit board; a light-emitting diode (LED) light sheet is further integrated on the circuit board; a plastic partition plate with the hollow middle is arranged in the hollow diffuser body and divides the hollow diffuser body into an upper part and a lower part; a ceramic atomizing sheet is installed on the middle hollow part of the plastic partition plate; the ceramic atomizing sheet is connected with the circuit board; one side wall of the hollow diffuser body is provided with an exhaust channel, and the exhaust channel penetrates the plastic partition plate to have a ventilation effect; and a cover with a vent hole is further arranged above the hollow diffuser body.

As a preferred embodiment:

the base is further provided with an atomization switch and an LED light switch;

the outer cover is made of hollow ceramics, glass, iron or a combination thereof, and is used for decoration;

a tooth-shaped silica gel ring is further arranged outside the hollow diffuser body, to enable the outer cover to cover the hollow diffuser body more tightly;

a plastic ring with a screw hole is arranged between the base and the hollow diffuser body, and the base is connected with the hollow diffuser body through a screw;

the base is provided with a power supply placing hole to be connected with an external transformer; and

a hollow fence for ventilation is further arranged on the lower portion of the base.

Through the technical solution, according to the atomizing aroma diffuser, when aromatherapy needs to be used, the outer cover and the cover above the hollow diffuser body are opened, essential oil and water are poured into the hollow diffuser body, the power supply is on, and the essential oil and the water are subjected to ultrasonic atomization through the ceramic atomizing sheet; a fan blows to discharge the atomized essential oil and water out of the vent hole through the exhaust channel, so that moisture and aroma can be added to a room. Meanwhile, the atomization switch on the lower portion of the base can control the fog amount and the LED light switch can control an LED light to change different colors, and the light penetrates the outer cover with a hollow pattern, so that a cozy home atmosphere can be created. The atomizing aroma diffuser is simple to operate, high in practicality and attractive in appearance.

### DESCRIPTION OF THE DRAWINGS

FIG. 1 is a schematic unfolded structure diagram of an atomizing aroma diffuser according to the present invention.

There is an outer cover 1, a hollow diffuser body 2, a base 3, a vent hole 4, a fan 5, a circuit board 6, a plastic partition plate 7, a ceramic atomizing sheet 8, a cover 9, a plastic ring 10, a silica gel ring 11, an atomizing switch 12, an LED light switch 13 and an exhaust channel 14.

### DETAILED DESCRIPTION OF PREFERRED EMBODIMENTS

The present invention is further described below in combination with a concrete embodiment.

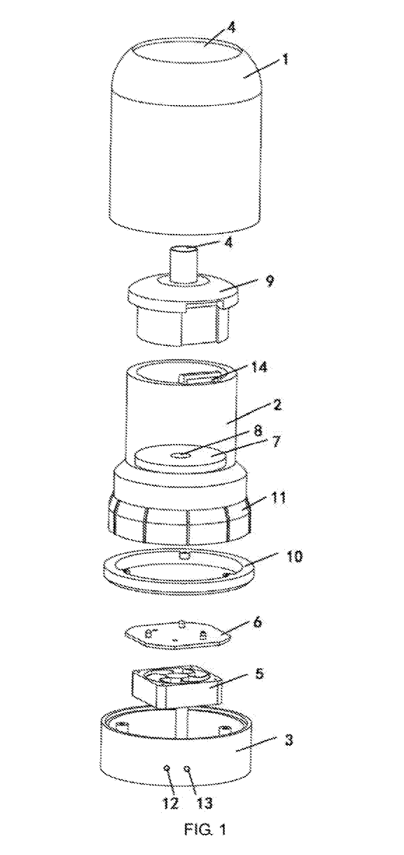

As shown in FIG. 1, as a preferred embodiment, an atomizing aroma diffuser comprises an outer cover 1, a hollow diffuser body 2 and a base 3, wherein the outer cover 1 covers the outside of the hollow diffuser body 2 to achieve a decoration effect; the upper surface of the outer cover 1 is provided with a vent hole 4; the base 3 is connected with the lower end of the hollow diffuser body 2; the base is provided with a fan 5 which is electrically connected with a circuit board 6; a light-emitting diode (LED) light sheet is further integrated on the circuit board 5; a plastic partition plate 7 with the hollow middle is arranged in the hollow diffuser body 2 and divides the hollow diffuser body into an upper part and a lower part, a ceramic atomizing sheet 8 is installed on the middle hollow part of the plastic partition plate 7; the ceramic atomizing sheet 8 is connected with the circuit board 6; and a cover 9 with a vent hole is further arranged above the hollow diffuser body 2.

As a concrete embodiment of the present invention, the base is further provided with an atomization switch 12 and an LED light switch 13. One side wall of the hollow diffuser body is provided with an exhaust channel 14, and the exhaust channel 14 penetrates the plastic partition plate to have a ventilation effect. The outer cover 1 can be made of hollow ceramics, glass, iron or a combination thereof, and is used for decoration. A plastic ring 10 with a screw hole is arranged between the base 3 and the hollow diffuser body 2, and the base is connected with the hollow diffuser body through a screw. Moreover, the base is provided with a power supply placing hole to be connected with an external transformer. A hollow fence for ventilation is further arranged on the lower portion of the base. A tooth-shaped silica gel ring 11 is further arranged outside the hollow diffuser body, to enable the outer cover 1 to cover the hollow diffuser body 2 more tightly.

When aromatherapy needs to be used, the outer cover 1 and the cover 9 above the hollow diffuser body are opened, essential oil and water are poured into the hollow diffuser body 2, the power supply is on, and the essential oil and the water are subjected to ultrasonic atomization through the ceramic atomizing sheet 8; and a fan 5 blows to discharge the atomized essential oil and water out of the vent hole 4 through the exhaust channel 14, so that moisture and aroma can be added to a room. The atomization switch 12 on the lower portion of the base can control the fog amount and the LED light switch 13 can control an LED light to change different colors, so that a cozy home atmosphere can be created.

I claim:

1. An atomizing aroma diffuser, comprising an outer cover, a hollow diffuser body and a base, wherein the outer cover covers the outside of the hollow diffuser body; the upper surface of the outer cover is provided with a vent hole; the outer cover is made of hollow ceramics, glass, iron or a combination thereof, and is used for decoration; the base is connected with the lower end of the hollow diffuser body; the base is provided with a fan which is electrically connected with a circuit board; a light-emitting diode (LED) light sheet is further integrated on the circuit board; a plastic partition plate with the hollow middle is arranged in the hollow diffuser body and divides the hollow diffuser body into an upper part and a lower part; a ceramic atomizing sheet is installed on the middle hollow part of the plastic partition plate and connected with the circuit board; one side wall of the hollow diffuser body is provided with an exhaust channel, and the exhaust channel penetrates the plastic partition plate; a cover with a vent hole is further arranged above the hollow diffuser body; and a tooth-shaped silica gel ring is further arranged outside the hollow diffuser body, to enable the outer cover to cover the hollow diffuser body more tightly.

2. The atomizing aroma diffuser according to claim 1, wherein the base is further provided with an atomization switch and an LED light switch.

3. The atomizing aroma diffuser according to claim 1, wherein a plastic ring with a screw hole is arranged between the base and the hollow diffuser body, and the base is connected with the hollow diffuser body through a screw.

4. The atomizing aroma diffuser according to claim 1, wherein the base is provided with a power supply placing hole to be connected with an external transformer.

5. The atomizing aroma diffuser according to claim 1, wherein a hollow fence for ventilation is further arranged on the lower portion of the base.
